(19) United States
(12) Patent Application Publication
(10) Pub. No.: US 2007/0049958 A1
(43) Pub. Date: Mar. 1, 2007

(54) Spiral cut curved blade

(75) Inventor: Kenneth M. Adams, Naples, FL (US)

Correspondence Address: DICKSTEIN SHAPIRO LLP, 1825 EYE STREET NW, Washington, DC, 20006-5403, US

(73) Assignee: Arthrex, Inc.

(21) Appl. No.: 11/512,300

(22) Filed: Aug. 30, 2006

Related U.S. Application Data
(60) Provisional application No. 60/712,172, filed on Aug. 30, 2005.

(51) Int. Cl. A61B 17/32

(52) U.S. Cl. 606/167

(57) ABSTRACT

An arthroscopic shaver with an outer tubular member and an inner tubular member rotatably disposed within the outer tubular member. The inner tubular member has a proximal end portion and a distal end portion, with a cutting means on the distal end portion. The inner tubular member has a plurality of helical segments or two sliced tubes of stainless steel wound in alternating opposite directions providing a more flexible configuration and better torque capability for the inner tubular member.

This application claims the benefit of U.S. Provisional Application Ser. No. 60/712,172, filed on Aug. 30, 2005, the entire disclosure of which is incorporated herein by reference.

### FIELD OF THE INVENTION

The present invention relates to the field of surgical cutting instruments and, more particularly, to instruments having relatively movable inner tubular members.

### BACKGROUND OF THE INVENTION

Surgical cutting instruments in which an inner member is rotated within an elongate tubular outer member are known in surgical procedures where access to the surgical site is via a narrow portal or passage. Typically, the tubular outer member has a distal end with an opening defining a cutting port or window. The inner member has a distal end with a cutting tip for engaging bodily tissue via the opening. Proximal ends of the inner and outer members commonly include hubs which attach to a handpiece having a motor for rotating the inner member relative to the outer member. The distal end of the inner member can have various configurations dependent upon the surgical procedure to be performed. Often the inner member is tubular so that the loose tissue resulting from a cutting, resecting or abrading procedure can be aspirated through the lumen of the inner member.

Although most surgical cutting instruments are straight, in many surgical procedures it is desirable for the cutting instruments to be bent or curved to access surgical sites which are generally not accessible with straight cutting instruments. For example, in arthroscopic knee surgery it is well known to use curved cutting instruments which can be positioned at various desired angles relative to the surface of the patella. While rotary tissue cutting instruments with curved or bendable shafts have been used for some time, these shafts typically employ a single spirally wound strip of material to impart flexibility while transmitting torque. Unfortunately, spirally wound shafts and couplings tend to unwind when rotated in a direction opposite their winding so that torque can only be transmitted efficiently in one direction.

Accordingly, there is a need to provide an improved flexible cutting instrument used in arthroscopic surgery, that can be rotated even if it is curved and without unwinding of the shafts when rotated in two directions. A surgical cutting instrument that has increased torque capability is also needed.

### BRIEF SUMMARY OF THE INVENTION

The present invention provides a surgical cutting instrument comprising an outer tubular member having a proximal end and a distal end. An inner tubular member includes a distal end portion having cutting means, and a proximal end portion: The inner tubular member is rotatably disposed within the outer tubular member.

The outer tubular member is constructed of a rigid material that is bent during fabrication. Once bent, the outer tubular member retains the selective bent configuration. The cutting means or cutter is rotatably disposed within the outer tubular member adjacent to an outer cutting aperture.

The inner member is flexible and is connected to the cutter. The inner tubular member comprises two sliced tubes of stainless steel, for example, with each of the two layers being sliced in opposite directions. The two sliced layers have diameters that allow sliding contact with a minimum of radial clearance between adjacent surfaces. The two sliced layers are wound in alternating opposite directions to form a flexible tube. Torque applied to the surgical cutting instrument will be transmitted by the two alternate layers trying to expand or unwind or trying to contract or wind up, providing a flexible transmission that is rotatable bidirectionally (clockwise and counter-clockwise).

These and other features and advantages of the invention will be more apparent from the following detailed description that is provided in connection with the accompanying drawings and illustrated exemplary embodiments of the invention.

### BRIEF DESCRIPTION OF THE DRAWINGS

FIG. 1 is a side view of the outer tubular member and laser sliced tube of the inner tubular member of a surgical cutting instrument of the present invention;

FIG. 2 is a side view of a laser sliced tube of the inner tubular member of FIG. 1;

FIG. 3 is a cross-sectional partial view of the sliced tube of the inner tubular member of FIG. 2;

FIG. 4 is another cross-sectional partial view of the sliced tube of the inner tubular member of FIG. 2;

FIG. 5 is a side view of a laser sliced tube of the inner tubular member in accordance with another embodiment of the present invention;

FIG. 6 is a side view of a laser sliced tube of the inner tubular member in accordance with another embodiment of the present invention; and

FIG. 7 is a side view of a laser sliced tube of the inner tubular member in accordance with another embodiment of the present invention.

### DETAILED DESCRIPTION OF THE INVENTION

The present invention provides an apparatus for arthroscopic surgery that utilizes a curved blade that can be rotated. The present invention also provides increased torque capability when undergoing arthroscopic surgery.

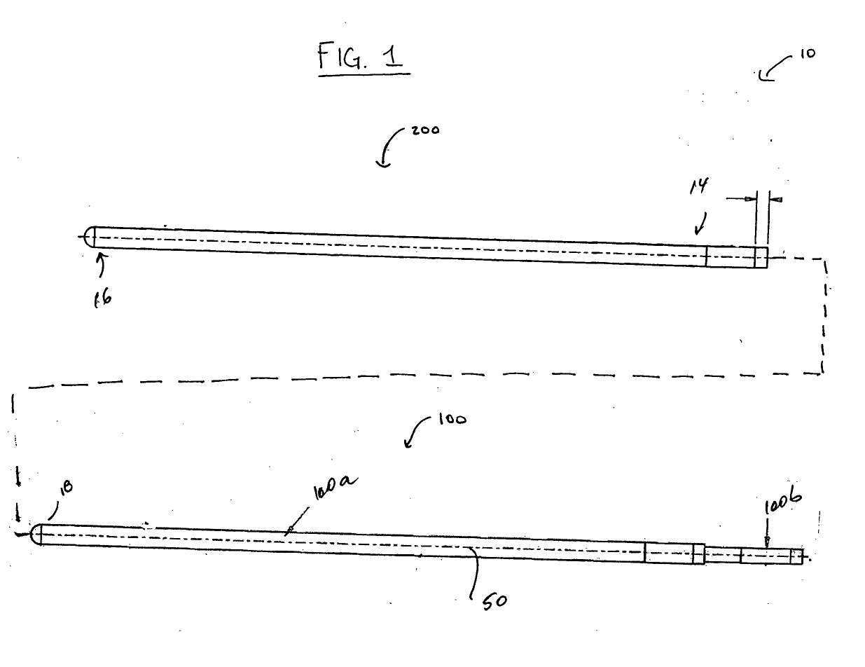

Referring now to the drawings, where like elements are designated by like reference numerals, FIGS. 1-6 illustrate an exemplary embodiment of inner tubular member 100 and outer tubular member 200 of a surgical cutting instrument 10 of the present invention, in which flexible inner tubular member 100 freely rotates within a rigid, bent outer tubular member 200.

Referring specifically to FIG. 1, the surgical cutting tool 10 of the present invention includes an inner tubular member 100 rotatably disposed within an outer tubular member 200, which has a proximal end 14 and a distal end 16. The outer tubular member 200 is elongated and has a distal end 16 having an opening facing upwardly which defines a cutting port or window (not shown). A tissue cutting means (not shown) is rotatably disposed within the outer tubular member 200.

The outer tubular member 200 is selectively bent during the fabrication of the tool. The outer tubular member 200 is of a material which, when bent, retains the bent configuration. An example of a suitable material for use in the fabrication of the outer tubular member 200 is stainless steel.

Inner tubular member 100 is disposed coaxially or concentrically within the outer tube 200. The inner tube 100 is elongated and has distal and proximal ends with a cutting means or cutter 52 (FIG. 5) disposed at the distal end portion 18. As detailed below, the inner tubular member 100 transmits rotational movement from a motor (not shown) to the cutting means 52. This facilitates cutting of the anatomical tissue. The cutting means 52 may have various configurations, known in the art, to cut the anatomical tissue. The inner tube 100 has a hollow cylindrical configuration—the lumen of the inner tubular member 100 provides an unimpeded path for fluid and debris to be aspirated away from the surgical site.

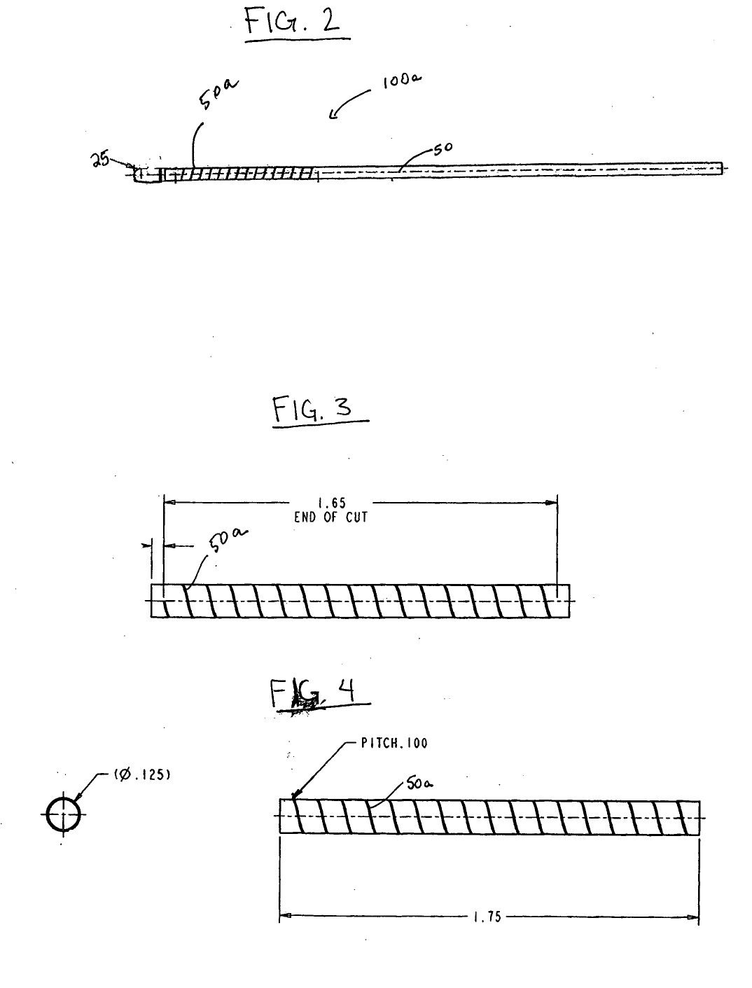

In a preferred embodiment, inner tube 100 comprises two coaxial tubes 100a (shown in FIG. 2), 100b (not shown), both having a hollow cylindrical configuration. Each of the two coaxial inner tubes 100a, 100b is sliced or cut to form a plurality of serially arranged, interconnected helical or spiral segments 50a (shown in FIG. 2), 50b (not shown) longitudinally spaced from one another lengthwise along each of their respective tubes 100a, 100b. Preferably, the inner tubes 100a, 100b are laser sliced or laser cut to form the spiral segments 50a, 50b.

The spiral segments 50a, 50b extend continuously in a helical or spiral path, i.e. an open path, along the cylindrical walls forming tubes 100a, 100b and about the central longitudinal axis 50 (FIG. 1) of tube 100, such that opposite ends of the helical cut do not meet. As illustrated in FIG. 2, the spiral segments 50a extend around the central longitudinal axis 50 of the first inner tube 100a in a first direction. The spiral segments 50b (not shown) extend about the central longitudinal axis 50 of the second inner tube 100b in a second direction, which is with a clockwise or right hand turn or slant looking from distal to proximal relative to the first direction.

In a preferred embodiment, the inner tube 100 is formed of a medically acceptable material such as stainless steel. The inner tube 100a is about 6.39 inches long and has an outer diameter of about 0.139 inches and an inner diameter of about 0.099 inches. This allows the inner tube 100 to be disposed tightly within the outer tube 200, which is about 5.60 inches long and has an outer diameter of about 0.165 inches and an inner diameter of about 0.145 inches. Helical segment 50a on inner tube 100a, as shown in FIG. 2, begins about 0.28 inches from the distal end of the elliptical tip 25 and extends proximally and terminates about 1.55 inches from the distal end of the elliptical tip 25.

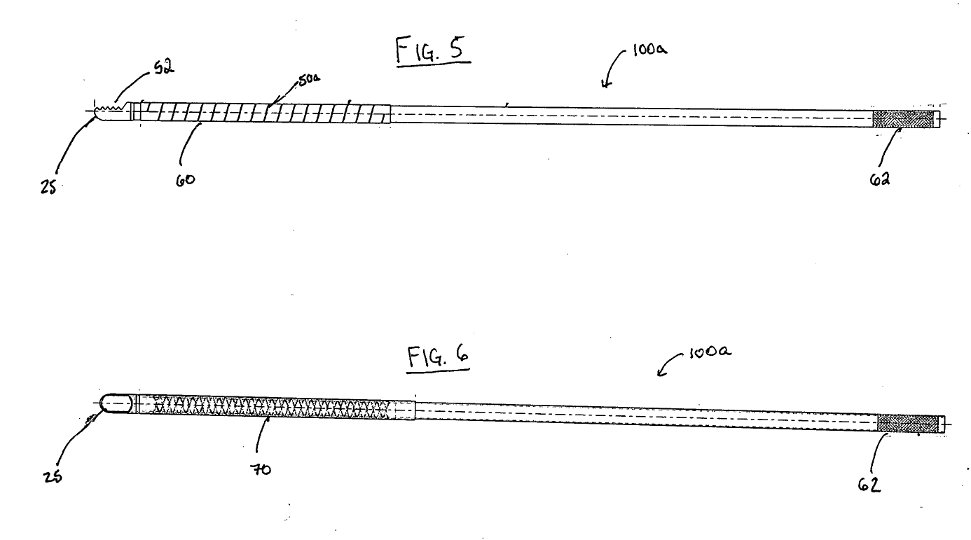

As illustrated in FIGS. 3-4, a laser cut sleeve is shown over the helical segments 50a. The sleeve begins about 0.28 inches from the distal end of the elliptical tip 25 (FIG. 2) and extends proximally about 1.65 inches to end about 1.75 inches from the distal end of the elliptical tip 25. As illustrated in FIG. 4, the inner diameter of the laser cut sleeve is 0.125 inches.

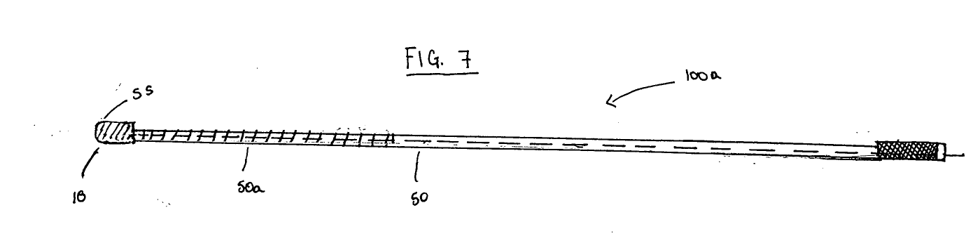

Referring to FIGS. 5-6, the sleeve, preferably formed of a polymer material, may be welded onto the inner tube 100a. Sleeve 60 is welded into place over the helical segments 50a on inner tube 100a. Both ends of sleeve 60 are laser welded into place. This sleeve 60 begins about 0.35 inches from the distal end of the elliptical tip 25 and extends proximally until about 1.9 inches from the distal end of the elliptical tip 25. Further, a shrink tube 70 is installed over sleeve 60 and is set into place by heat-shrinking or shrink-wrapping. The shrink tube 70 begins about 0.30 inches from the distal end of the elliptical tip 25 and extends proximally until about 2.1 inches from the distal end of the elliptical tip 25. A raised diamond knurl 62 at the proximal end of the inner tube 100a is a point of attachment for the inner tube 100 with the outer tube 200.

Referring to FIG. 7, in another preferred embodiment, the inner tube 100 has an abrading element 55 or burr disposed at the distal end portion 18 to abrade the tissue. As discussed above, the inner tubular member 100 transmits rotational movement from a motor (not shown) to the abrader 55. The abrader 55 is used for abrading or shaping hard tissue such as bone or cartilage by use of the rotating abrading head. As the tissue is being abraded, debris and fluid are usually aspirated. As discussed above, spiral segments 50a, 50b (not shown) extend continuously in a helical or spiral path along the cylindrical walls forming tubes 100a, 100b (not shown) and about the central longitudinal axis 50 of tube 100, such that opposite ends of the helical cut do not meet.

By providing the two sliced inner layers wound in alternating opposite directions, the inner tube becomes flexible and can retain its flexible configuration even when inserted through a bent, yet rigid, outer tube. Torque applied to the surgical cutting instrument will be transmitted by the two alternate layers trying to expand or unwind or trying to contract or wind up, providing a flexible transmission. Further, the sliced inner layers prevent the inner tube from expanding, unwinding, or contracting from within the outer tube when the arthroscopic shaver is rotating in an oscillating mode or bi-directionally.

The above description and drawings illustrate preferred embodiments which achieve the objects, features, and advantages of the present invention. It is not intended that the present invention be limited to the illustrated embodiments, but rather only by the appended claims. Any modification of the present invention which comes within the spirit and scope of the following claims should be considered part of the present invention.

What is claimed as new and desired to be protected by Letters Patent of the United States is:

1. An instrument for removing anatomical tissue, comprising:
an outer tubular member having a distal end, a proximal end, and a fixed bend; and
an inner tubular member disposed within said outer tubular member and comprising at least two tubes having helical segments formed in alternating opposite directions.

2. The instrument according to claim 1, wherein the inner tubular member is rotatably disposed within said outer tubular member.

3. The instrument according to claim 1, wherein said inner tubular member includes a distal end portion having a cutting means.

4. The instrument according to claim 3, wherein said cutting means is a shaver blade.

5. The instrument according to claim 1, wherein said inner tubular member includes a distal end portion having an abrading element.

6. The instrument according to claim 1, wherein said helical segments are formed about a central longitudinal axis of said inner tubular member.

7. The instrument according to claim 1, wherein said helical segments are laser sliced on said two tubes.

8. The instrument according to claim 1, wherein said outer and inner tubular members are stainless steel.

9. The instrument according to claim 1, wherein said outer and inner tubular members are a hollow cylindrical configuration.

10. The instrument according to claim 1, wherein a sleeve is attached to said inner tubular member.

11. The instrument according to claim 10, wherein said sleeve is laser cut.

12. The instrument according to claim 10, wherein said sleeve is a polymer.

13. The instrument according to claim 10, wherein said sleeve is welded onto said inner tubular member.
